(19) United States
(12) Patent Application Publication
(10) Pub. No.: US 2022/0192602 A1
(43) Pub. Date: Jun. 23, 2022

(54) SMART CONTROLLER INCLUDING SENSOR, AND PORTABLE TERMINAL CASE INCLUDING SENSOR

(71) Applicant: ITVERS CO.,LTD, Seongnam-si, Gyeonggi-do (KR)

(72) Inventor: Youn Soo KIM, Seoul (KR)

(21) Appl. No.: 17/601,424

(22) PCT Filed: Apr. 3, 2020
(86) PCT No.: PCT/KR2020/004576

§ 371 Date: Oct. 4, 2021

(30) Foreign Application Priority Data
Apr. 4, 2019 (KR) 10-2019-0039500

(51) Int. Cl. A61B 5/00; A61B 5/024; A61B 5/01; A61B 5/332; H04B 1/3888; H04M 1/72409; H04M 1/04

(52) U.S. Cl. CPC A61B 5/6898; A61B 5/02444; A61B 5/01; H04M 1/04; A61B 5/4872; H04B 1/3888; H04M 1/724092; A61B 5/332

(57) ABSTRACT

A smart controller with a sensor, according to one embodiment of the present inventive concept, communicates wirelessly with a mobile terminal and has a sensor for providing, to the mobile terminal, biometric information of a user or external environmental information, the smart controller comprising: a body; an input button unit which is arranged on the upper surface or a side surface of the body and which generates a control signal for the mobile terminal by means of operation by the user; a sensor for detecting biometric information of the user or external environmental information; and a control unit which is arranged inside the body and which receives an input of a detection value of the sensor and delivers same to the mobile terminal.

### TECHNICAL FIELD

The present disclosure relates to a controller, and more specifically, to a smart controller that is capable of generating an input signal for a mobile terminal such as a smartphone or a wireless communication device and detecting biometric information of a user or external environmental information to provide the detected result for the user.

### BACKGROUND ART

Recently, a lot of mobile terminals such as smartphones are launched.

Conventional smartphones are easy to carry around and have small liquid crystal displays to faithfully perform their inherent call functions, but many users prefer obtaining lots of information on a wide screen to easily carry the smartphones. Accordingly, the smartphones with large liquid crystal displays have been steadily increased.

The enlargement of the size of the smartphone satisfies the user's visual comfort, but it is hard to control the smartphone with one hand.

Further, most of inputs on the smartphone are generated through a touchscreen, and if a game is played through the smartphone, it is not easy to accurately control playing the game. If a control button provided on the touchscreen is pressed by the user's hand, besides, the control button or user's hand may hide the display of the smartphone.

### DISCLOSURE

### Technical Problem

To solve the above-mentioned problems occurring in the related art, accordingly, a smart controller with a sensor and a mobile terminal case with a sensor according to the present inventive concept have been made with the following objects.

It is an object of the present inventive concept to provide a smart controller and a mobile terminal case that are capable of freely and easily controlling a mobile terminal with a large size display through only one hand of a user.

It is another object of the present inventive concept to provide a smart controller and a mobile terminal case that are capable of providing a user's biometric information and external environmental information through a mobile terminal of the user.

The technical problems to be achieved through the present inventive concept are not limited as mentioned above, and other technical problems not mentioned herein will be obviously understood by one of ordinary skill in the art through the following description.

### Technical Solution

To accomplish the above-mentioned objects, according to one aspect of the present inventive concept, there is provided a smart controller performing wireless communication with a mobile terminal and providing, to the mobile terminal, biometric information of a user of the mobile terminal or external environmental information, the smart controller including: a body; an input button unit disposed on the upper or side surface of the body to generate a control signal for the mobile terminal through the control of the user; a sensor for detecting the biometric information of the user or the external environmental information around the user; and a control unit disposed inside the body to receive a detection value from the sensor and to thus transmit the received detection value to the mobile terminal.

Desirably, the sensor may be a bio sensor for detecting the biometric information of the user, the bio sensor being adapted to measure at least one of blood glucose, hemoglobin concentration, heart rate, body temperature, electrocardiogram, cholesterol level, and body fat rate of the user.

Desirably, the sensor may be an eco sensor for detecting the environmental information around the user of the mobile terminal, the eco sensor being adapted to measure at least one of radioactive concentration, ozone concentration, carbon monoxide concentration, carbon dioxide concentration, fine dust concentration, and ultraviolet index.

Desirably, the body has an accommodation portion for accommodating the sensor therein, and the sensor is detachably attached to the accommodation portion.

Desirably, the input button unit and the sensor are formed integrally with each other.

Desirably, the mobile terminal has an application installed therein to perform at least one of functions of controlling the operation of the sensor and outputting the detection value of the sensor.

Desirably, if the operation of the sensor is requested from the user through the application, the mobile terminal transmits a sensor-operating signal to the control unit of the smart controller, and the control unit operates the sensor in response to the sensor-operating signal.

Desirably, the control unit transmits the detection value of the sensor to the mobile terminal, and based on the detection value of the sensor, the application outputs a detection result to a display of the mobile terminal.

Desirably, the input button unit may include a sensing button for operating the sensor.

Desirably, if the sensing button operates, the control unit allows the application preinstalled in the mobile terminal and the sensor to operate.

Desirably, if the sensing button operates, the sensor detects the user's biometric information or external environmental information a plurality of times.

Desirably, if a deviation among the values detected the plurality of times by the sensor is greater than a preset value, the control unit allows the sensor to operate again.

Desirably, the smart controller may further include a support unit rotatably disposed on a top or side surface of the body to support the mobile terminal.

Desirably, the smart controller may further include a connection member disposed on the underside of the body in such a manner as to be attached to the underside of the mobile terminal or the bottom surface of a mobile terminal case for accommodating the mobile terminal.

To accomplish the above-mentioned objects, according to another aspect of the present inventive concept, there is provided a mobile terminal case accommodating a mobile terminal therein and performing wireless communication with the mobile terminal, the mobile terminal case including: an accommodation portion formed on the front surface thereof to accommodate the mobile terminal therein; an input button unit disposed on the back surface of the mobile terminal case which faces the accommodation portion to generate a control signal for the mobile terminal through a user's control; a sensor disposed on the back surface of the mobile terminal case which faces the accommodation portion to detect the user's biometric information or external environmental information; and a control unit adapted to receive a detection value from the sensor and to thus transmit the received detection value to the mobile terminal.

Desirably, the sensor may be a bio sensor for detecting the biometric information of the user, the bio sensor being adapted to measure at least one of blood glucose, hemoglobin concentration, heart rate, body temperature, electrocardiogram, cholesterol level, and body fat rate of the user.

Desirably, the sensor may be an eco sensor for detecting the environmental information around the user of the mobile terminal, the eco sensor being adapted to measure at least one of radioactive concentration, ozone concentration, carbon monoxide concentration, carbon dioxide concentration, fine dust concentration, and ultraviolet index.

Desirably, the mobile terminal case may further include a support unit rotatably coupled to the back surface facing the accommodation portion to support the mobile terminal.

### Effective Advantages of the Inventive Concept

According to the present inventive concept, each of the smart controller and the mobile terminal case may include the input button unit for controlling at least one of the smartphone as the mobile terminal and the wireless communication device, so that the user can control the mobile terminal, while supporting the mobile terminal with one hand, thereby improving the conveniences of the use of the mobile terminal.

Moreover, each of the smart controller and the mobile terminal case may include the bio sensor for detecting the biometric information of the mobile terminal user or the econ sensor for detecting the external environmental information around the mobile terminal user, so that through the mobile terminal, the user can easily check the biometric information such as his or her blood glucose or the environmental information such as a current fine dust concentration.

The effectiveness of the invention is not limited as mentioned above, and it should be understood to those skilled in the art that the effectiveness of the invention may include another effectiveness as not mentioned above from the detailed description of the present inventive concept.

### DESCRIPTION OF DRAWINGS

FIG. 1 is a perspective view showing a smart controller with a sensor according to an embodiment of the present inventive concept.

FIGS. 2 and 3 are sectional views showing a smart button capable of being used as a main button part in the smart controller according to the embodiment of the present inventive concept.

FIG. 4 is an exemplary view showing an example of using a mobile terminal case with a sensor according to another embodiment of the present inventive concept.

FIG. 5 is a front view showing the mobile terminal case with a sensor according to another embodiment of the present inventive concept.

FIG. 6 is a rear view showing the mobile terminal case with a sensor according to another embodiment of the present inventive concept.

### DETAILED DESCRIPTION

Hereinafter, embodiments of the present inventive concept will be explained in detail with reference to the attached drawings wherein the corresponding parts in the embodiments of the present inventive concept are indicated by corresponding reference numerals and the repeated explanation on the corresponding parts will be avoided.

If it is determined that the detailed explanation on the well-known technology related to the present inventive concept makes the scope of the present inventive concept not clear, the explanation will be avoided for the brevity of the description. In addition, the embodiments may be embodied in different forms and should not be construed as limited to the embodiments set forth herein but may be modified and variously implemented by those skilled in the art.

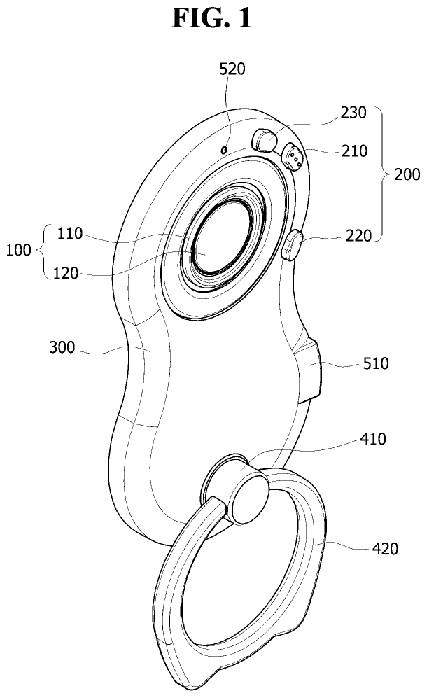

Hereinafter, a smart controller with a sensor according to an embodiment of the present inventive concept will be explained with reference to FIGS. 1 to 3.

The smart controller with a sensor according to an embodiment of the present inventive concept is capable of controlling a mobile terminal. In this case, the smart controller controls the mobile terminal through wired communication with the mobile terminal, desirably through wireless communication such as Bluetooth with the mobile terminal.

As shown in FIG. 1, the smart controller includes a main button unit 100 and an auxiliary button unit 200 as an input button unit for generating various control signals for the mobile terminal.

As shown in FIG. 1, the main button unit 100 largely includes a direction button 110 and a selection button 120.

The direction button 110 functions to generate a first signal related to directions through lateral pressurization, and the selection button 120 is located at the center or one side of the direction button 110 and functions to generate a second signal through downward pressurization.

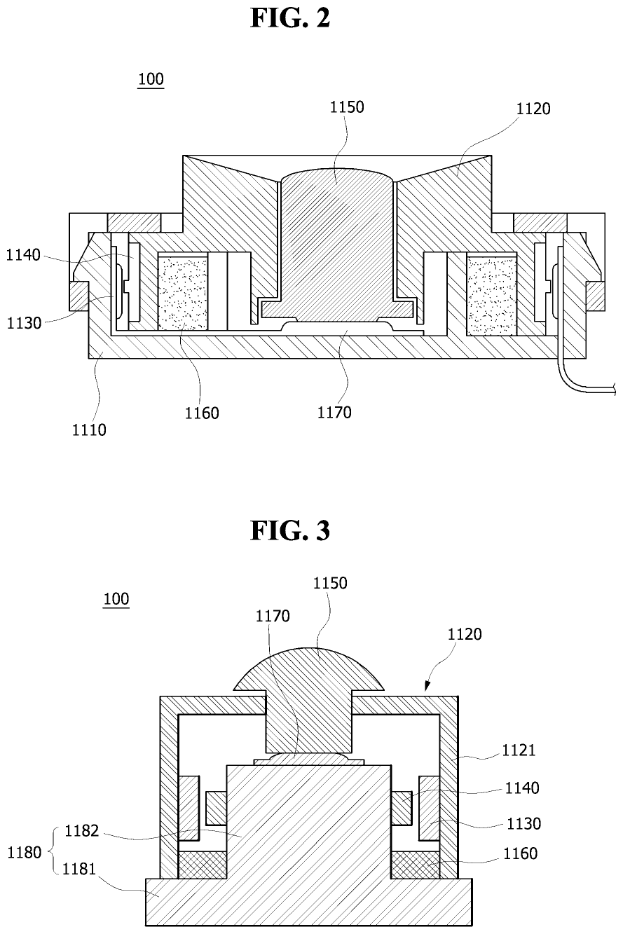

Further, the main button unit 100 may be adopted as smart buttons as shown in FIGS. 2 and 3, and through the smart buttons, the structure and function of the main button unit 100 can be made and provided.

The main button unit 100, which is provided for the smart controller with a sensor according to one embodiment of the present inventive concept, largely includes a housing 1110, a slide button 1120, a sensor 1130, and a contact 1140.

The housing 1110 has an inner space whose top is open.

The slide button 1120 is seated onto the inner space formed in the housing 1110 and movable by a given distance in front, back, left and right directions, while being not fixed to the inner space of the housing 1110.

The sensor 1130 is located on one surface of the outer peripheral surface of the slide button 1120 and the inner peripheral surface of the housing 1110, and the contact 1140 is located on the other surface of the outer peripheral surface of the slide button 1120 and the inner peripheral surface of the housing 1110.

In specific, the sensor 1130 is formed of a flexible printed circuit board having two or more terminal switches, and in this case, the terminal switches function as the sensor 1130.

The contact 1140 is desirably formed of a conductor, and when the slide button 1120 is pushed laterally, accordingly, the contact 1140 comes into contact with the sensor 1130 located in the direction to which the slide button 1120 moves, so that an input signal assigned to the sensor 1130 contacted with the contact 1140 is generated.

A slide type input button for generating an input signal through the lateral pressurization of the button, as mentioned above, has been suggested with various structures, and according to conventional slide type input buttons, in this case, pressurization sensors for sensing the lateral pressurization have to be necessarily needed, thereby undesirably raising a manufacturing cost of a signal input device.

In specific, if the slide type input button is configured to generate different kinds of input signals according to the slide directions thereof, the pressurization sensors have to be mounted correspondingly to respective directions to which pressurizing forces are applied, thereby making the manufacturing cost problem more serious.

According to one example of the main button unit 100 applied to the smart controller of the present inventive concept, however, different kinds of input signals may be generated according to the directions to which the pressurizing forces are applied in lateral directions, without any separate pressurization sensors.

As the slide button 1120 is pushed to move toward any one side direction, further, the contact 1140 is connected to the sensor 1130, and after that, the pressurizing force applied to the slide button 1120 is released. However, in this case, if the connected state of the sensor 1130 is still kept, the input signal may be consistently generated from the sensor 1130, irrespective of a user's intention.

To solve such a problem, the main button unit 100 further includes an elastic member 1160 for returning the slide button 1120 to its original position to allow the sensor 1130 and the contact 1140 to be separated from each other when the slide button 1120 moves in the lateral directions.

When the external force applied in the lateral directions to the slide button 1120 is released, accordingly, the slide button 1120 is returned to its original position through the elastic force of the elastic member 1160, thereby providing a state where the sensor 1130 and the contact 1140 are separated from each other, that is, where no input signal is generated.

According to one example of the main button unit 100 applied to the smart controller of the present inventive concept, as shown in FIG. 2, the main button unit 100 further includes a switch 1170 disposed on the bottom of the housing and a push button 1150 disposed on top of the switch 1170.

The push button 1150 passes through the slide button 1120 and is exposed upwardly from the slide button 1120 to the outside, while having the underside seated onto the switch 1170, and when a downward pressurizing force is applied to the push button 1150, the push button 1150 is pushed downwardly to pressurize the switch 1170, thereby generating a signal different from the signal generated by the movement of the slide button 1120.

Accordingly, various signals may be generated by the combination between the operations of the slide button 1120 and the push button 1150, and in this case, the slide button 1120 and the push button 1150 correspond to the direction button 110 and the selection button 120 of the smart controller according to one embodiment of the present inventive concept.

Hereinafter, another example of the main button unit 100 applied to the smart controller according to one embodiment of the present inventive concept will be explained with reference to FIG. 3.

The main button unit 100, which is provided for the smart controller according to one embodiment of the present inventive concept, largely includes a base member 1180, a slide button 1120, a sensor unit 1130, and a contact 1140.

The base member 1180 includes a bottom surface 1181 and a protrusion 1182 protruding upwardly from the center of the bottom surface 1181.

The slide button 1120 is located above the protrusion 1182 and has a downward side wall 1121 extended downwardly therefrom to surround the protrusion 1182.

Further, as shown in FIG. 3, the sensor unit 1130 is disposed on the outer peripheral surface of the protrusion 1182, and the contact 1140 is located on the corresponding area of the inner peripheral surface of the downward side wall 1121 to the area of the outer peripheral surface of the protrusion 1182 on which the sensor unit 1130 is located.

Contrarily, the sensor unit 1130 is disposed on the inner peripheral surface of the downward side wall 1121, and the contact 1140 is located on the corresponding area of the outer peripheral surface of the protrusion 1182 to the area of the inner peripheral surface of the downward side wall 1121 on which the sensor unit 1130 is located.

Under the above-mentioned configuration, if the slide button 1120 moves in a lateral direction, the sensor unit 1130 and the contact 1140 come into contact with each other to generate a signal. In specific, the slide button 1120 does not move in up and down directions but moves to a given distance in front, back, left and right directions, that is, in every direction. When the slide button 1120 is pushed in a lateral direction by the user, the contact 1140 comes into contact with the sensor unit 1130 located in the direction to which the slide button 1120 moves, so that the input signal related to the corresponding sensor of a plurality of sensors formed in the sensor unit 1130 is generated.

Accordingly, the slide button 1120 has to be configured to move in front, back, left and right directions, without rotating. If the slide button 1120 can rotate, the contact 1140 may not come into contact with the sensor desired by the user, so that undesirably, an input signal that is not desired by the user may be generated.

In addition, a touch sensor or tactile sensor may be used as the sensor unit 1130, but in consideration of manufacturing cost reduction and product lightness, desirably, a flexible printed circuit board is used as the sensor unit 1130.

That is, at least one or more sensors are located on the flexible printed circuit board, and the sensors are terminal switches each having a positive terminal and a negative terminal. The contact 1140 is formed of a conductor, and accordingly, when the slide button 1120 moves in a lateral direction, the positive terminal and the negative terminal of the terminal switch can come into contact with the contact 1140, so that the positive terminal and the negative terminal are short-circuited, thereby generating an input signal.

The terminal switch is configured to allow the positive terminal and the negative terminal to be spaced apart from each other side by side, and further, the positive terminal and the negative terminal have portions arranged alternately in a horizontal direction, that is, upward and downward concave and convex patterns arranged to alternately engage with each other. However, it is possible that the concave and convex portions of the positive terminal and the negative terminal may be arranged to alternately engage with each other, while being kept spaced apart from each other by a fine distance.

Further, only the contact portion of the contact 1140 may protrude outwardly so that the short circuit between the positive terminal and the negative terminal can be ensured.

In addition, the main button unit 100 further includes an elastic member 1160 for returning the slide button 1120 to its original position when the slide button 1120 moves in a lateral direction, and the elastic member 1160 is formed of an elastic material such as a carbon sponge, silicone, and the like so that when the slide button 1120 moves in a lateral direction, the elastic member 1160 is compressed to apply a returning elastic force to the slide button 1120.

Accordingly, various signals may be generated by the combination between the operations of the slide button 1120 and the push button 1150, and in this case, the slide button 1120 and the push button 1150 correspond to the direction button 110 and the selection button 120 of the smart controller according to one embodiment of the present inventive concept.

On the other hand, the smart controller according to one embodiment of the present inventive concept further includes the auxiliary button unit 200 provided separately from the main button unit 100, and the auxiliary button unit 200 includes at least one button for changing an operating mode of the main button unit 100 or performing a specific function of a mobile terminal 10.

In specific, the auxiliary button unit 200 performs a function of changing an operating mode, that is, the conversion into a keyboard and mouth mode of the main button unit 100, performs a function of controlling call volume, and performs a function of executing or controlling a pre-set specific application.

Further, the smart controller according to one embodiment of the present inventive concept includes a body 300 for accommodating the main button unit 100 and the auxiliary button unit 200 therein and a connection member disposed on the underside of the body 300 and attached to the underside of the mobile terminal or the bottom surface of the case for accommodating the mobile terminal.

Further, the smart controller according to one embodiment of the present inventive concept includes a support unit disposed on the body 300 to supportingly locate the mobile terminal on a table or floor.

The support unit is located on top of the body 300 and spaced apart from the main button unit 100 and the auxiliary button unit 200 by a given distance, so that the user's finger is inserted into the support unit to allow the main button unit 100 or the auxiliary button unit 200 to be controlled by his or her finger inserted.

As shown in FIG. 1, the support unit includes a connection member 410 attached to top of the body 300 in such a manner as to be rotatable in left and right directions and a ring member 420 coupled to the connection member 410 in such a manner as to be rotatable in up and down directions.

As a result, the ring member 420 is rotatable around the body 300 in up, down, left and right directions.

Otherwise, unlike the support unit as shown in FIG. 1, the support unit may have one end and the other end connected to one surface and the other surface of the body 300 so that the support unit may be rotatable around the body 300 in up and down directions.

Through the support unit as mentioned above, the mobile terminal can be slantly erected on the table or floor, and accordingly, it is possible for the user to easily see videos through the display of the mobile terminal, without any separate stand.

As mentioned above, the support unit has the shape of a ring, and accordingly, it is possible for the user to control the mobile terminal with one hand through his or her index or middle finger.

Moreover, the smart controller according to one embodiment of the present inventive concept includes a battery for supplying power, and the battery is chargeable. To do this, the body 300 has a connector terminal 510 adapted to charge the battery.

The connector terminal 510 is the same as of the mobile terminal in or to which the smart controller is accommodated or attached, thereby desirably improving conveniences for the user.

Instead of the connector terminal 510 for wired charging, further, a receiving coil for wireless charging may be disposed inside the body 300 to wirelessly receive power from an external charging device having a transmitting coil.

Further, the smart controller according to one embodiment of the present inventive concept includes a light emitting diode (LED) 520 for checking an operating state thereof.

On the other hand, unlike the configuration as shown in FIG. 1, the smart controller according to one embodiment of the present inventive concept may have two bodies.

For example, the smart controller may have a first body for accommodating the main button unit 100, the auxiliary button unit 200, and a control unit for generating control signals for the mobile terminal and a second body disposed separately from the first body and having a connection unit for connecting the mobile terminal 10 and the support unit attached thereto.

In this case, the second body has an accommodation portion for accommodating the first body therein and is detachable from the first body. If necessary, accordingly, the first body is attached to the underside of the mobile terminal to control the mobile terminal, and otherwise, the first body and the second body may be separated from each other to remotely control the mobile terminal.

Further, the smart controller according to one embodiment of the present inventive concept includes a sensor for detecting the biometric information of the user of the mobile terminal or external environmental information around the user.

The sensor may be a bio sensor for detecting the biometric information of the user, and desirably, the bio sensor measures at least one of the blood glucose, hemoglobin concentration, heart rate, body temperature, electrocardiogram, cholesterol level, and body fat rate of the user. In specific, the sensor for detecting the body temperature of the user may be a contact or non-contact type sensor.

Moreover, the sensor may be an eco sensor for detecting the environmental information around the user of the mobile terminal, and desirably, the eco sensor measures at least one of radioactive concentration, ozone concentration, carbon monoxide concentration, carbon dioxide concentration, fine dust concentration, and ultraviolet index.

Further, the body 300 has an accommodation portion for accommodating the sensor therein, and the sensor is detachably attached to the accommodation portion. Accordingly, the sensors corresponding to the information to be measured by the user according to his or her situation may be easily changed to one another.

Also, the sensor may be disposed integrally with the input button unit. In specific, the sensor may be provided separately from the main button unit 100 and the auxiliary button unit 200, and otherwise, the sensor may be provided on a portion of the selection button 120 of the main button unit 100, that is, on top of the selection button 120. Further, the sensor may be provided on a portion of the direction button 110.

Moreover, if the body 300 of the smart controller according to one embodiment of the present inventive concept includes the first body and the second body, the input button unit, the sensor, and the control unit are desirably provided on the first body.

Further, the input button unit of the smart controller according to one embodiment of the present inventive concept includes a sensing button for operating the sensor, and if the sensing button operates, the control unit allows the sensor to detect the user's biometric information or external environmental information around the user. Simultaneously, the control unit allows the application preinstalled in the mobile terminal to be executed.

For example, it is assumed that the sensor is an electrocardiogram sensor for measuring the user's electrocardiogram, and if the sensing button operates by the user, accordingly, the control unit allows the electrocardiogram sensor to measure the user's electrocardiogram. Simultaneously, the control unit executes the application related to electrocardiogram that is installed in the mobile terminal. Through the operation of the sensing button, as a result, the user can measure his or her electrocardiogram and check his or her current electrocardiogram through the display of the mobile terminal.

If the sensing button operates by the user, further, the control unit first checks the kind of sensor mounted and thus allows the mobile terminal to execute the application corresponding to the sensor mounted. Next, it is possible that the sensor mounted operates together with a guide message for notifying the user of biometric information sensing or environmental information sensing, which is produced from the application.

In specific, the sensing button may be a specific button included in the auxiliary button unit 200, and otherwise, the sensing button may be the selection button 120 of the main button unit 100.

If the sensing button is the selection button 120, the sensor is disposed on top of the selection button 120, and the push button under the selection button 120 is the sensing button, thereby improving conveniences for the user.

In addition to the method of operating the sensor through the sensing button disposed on the smart controller, the sensor may operate using an application preinstalled in the user's mobile terminal.

The application is installed in the mobile terminal and performs at least one of functions of controlling the operation of the sensor and outputting the detection value of the sensor.

In this case, if the operation of the sensor is requested by the user through the application installed in the user's mobile terminal after the application has been executed, the mobile terminal transmits a sensor-operating signal to the control unit of the smart controller, and the control unit operates the sensor in response to the sensor-operating signal.

After that, if the sensor detects the user's biometric information or the external environmental information and acquires the detection value, the control unit transmits the acquired detection value to the mobile terminal, and the application outputs a detection result such as detection value and evaluation to the display of the mobile terminal.

For example, if the application installed in the user's mobile terminal is executed, an icon for operating the sensor is outputted on the display of the mobile terminal, and if the icon is touched by the user, guide information for sensing is outputted to the display. Simultaneously, the mobile terminal transmits the sensor-operating signal to the control unit of the smart controller.

The control unit operates the sensor in response to the sensor-operating signal, and if the user's biometric information or the external environmental information is detected through the operation of the sensor, the control unit transmits the detection result to the mobile terminal to thus allow the detection result to be outputted to the display of the mobile terminal through the processing of the application.

To ensure accurate measurement of the sensor, the smart controller according to one embodiment of the present inventive concept is configured to allow the sensor to detect the user's biometric information or the external environmental information a plurality of times if the sensing button operates by the user, and if a deviation among the values detected the plurality of times by the sensor is greater than a preset value, the control unit allows the sensor to operate again.

For example, it is assumed that the sensor is a bio sensor that comes into contact with the user's finger to detect the body temperature of the mobile terminal user, and if the user's finger is not placed accurately on the sensor or not placed on the sensor for the operating time of the sensor, accordingly, the sensor fails to accurately detect the user's body temperature.

To prevent occurrence of such a problem, the sensor detects the user's body temperature a plurality of times within a predetermined time.

In detail, the sensor is set to detect the user's body temperature three times within a predetermined time, and if the first, second and third body temperatures measured sequentially by the sensor are 42.3° C., 36.9° C. and 36.8° C., the control unit determines that the user's finger is not placed accurately on the sensor so that the sensor detects the user's body temperature again.

As a result, the accuracy of the measurement of the user's biometric information or the external environmental information can be ensured.

To prevent the measurement of the user's biometric information or the external environmental information from being erroneous due to the failure of the sensor, further, the smart controller according to one embodiment of the present inventive concept includes a plurality of sensors, desirably, three or more sensors.

If a deviation among the values detected by the plurality of sensors is less than a preset value, the average value of the values detected by the plurality of sensors is outputted as the detection result, and if a deviation among the values detected by the plurality of sensors is greater than a preset value, the control unit determines that at least one of the plurality of sensors is not working.

To prevent the failure of the sensor from being determined quickly just by one time measurement in determining whether the sensor is not working, however, if the deviation among the values detected by the plurality of sensors is greater than the preset value, the control unit desirably operates the plurality of sensors again.

The plurality of sensors operates again by the control of the control unit, but if a difference between a value detected by any one sensor and values detected by other sensors is greater than a preset value, it is determined that the corresponding sensor is not working.

The determination of the failure of the sensor based on the measurement result of the sensor is carried out by the control unit of the smart controller, but of course, the determination may be carried out by the mobile terminal.

In this case, if the application of the mobile terminal determines that the value detected by the sensor is greater than a preset value, the mobile terminal transmits warning information to the control unit of the smart controller.

In specific, the smart controller according to one embodiment of the present inventive concept further includes a warning unit operating based on the warning information, and the warning unit includes at least one of a light emitting element and a speaker, so that if the sensor is not working, the user can easily recognize the problem through the operation of the warning unit.

While the warning unit is being not mounted in the smart controller, of course, it is possible that the related application installed on the mobile terminal transmits the warning message to the user through the display, speaker, and the like mounted on the mobile terminal.

If three or more sensors are provided, the warning information is not generated by the failure of only one sensor, and through the measured results of the two normal sensors, the user's biometric information and the external environmental information can be detected.

Moreover, the sensors may be provided as a sensing module detachably attached to the body 300, and the sensing module has a connection terminal corresponding to the connector terminal 510 disposed on the body 300.

That is, the connection terminal is fastened to the connector terminal 510, and accordingly, the value detected by the sensing module is transmitted to the control unit located inside the body 300, so that various kinds of sensing modules can be easily detachably attached to the smart controller, thereby allowing various biometric information and external environmental information to be detected using the single smart controller.

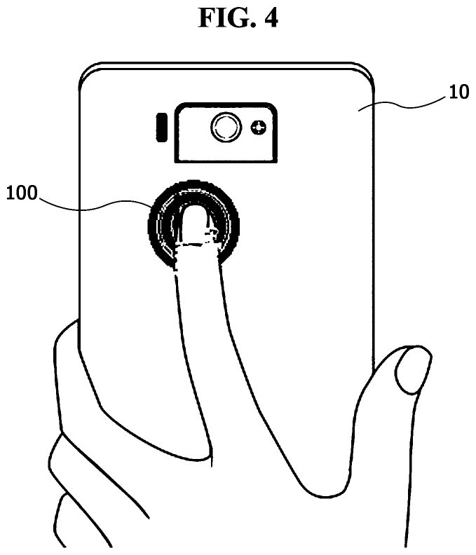

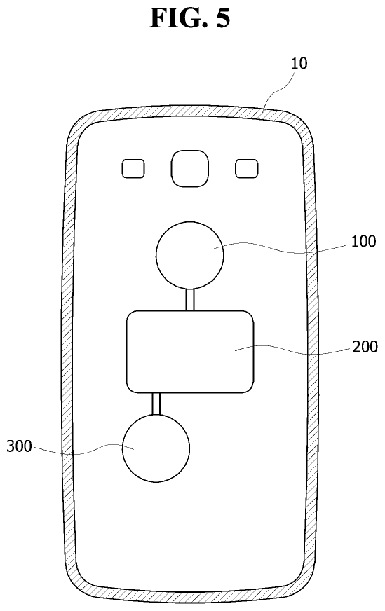

Hereinafter, an explanation of a mobile terminal case with a sensor according to another embodiment of the present inventive concept will be given with reference to FIGS. 4 to 6, and the explanation of the parts of the mobile terminal case corresponding to those of the smart controller according to one embodiment of the present inventive concept will be avoided for the brevity of the description.

The mobile terminal case according to another embodiment of the present inventive concept relates to the mobile terminal case that is adapted to accommodate a mobile terminal therein to perform wireless communication with the mobile terminal and includes an accommodation portion, an input button unit, a sensor, and a control unit.

The accommodation portion is formed on the front surface of the mobile terminal case to accommodate the mobile terminal therein, and the input button unit is disposed on the back surface of the mobile terminal case which faces the accommodation portion to generate a control signal for the mobile terminal through a user's control.

Like the smart controller according to one embodiment of the present inventive concept, the input button unit includes a main button unit 100 and an auxiliary button unit 400, and since an explanation of the main button unit 100 and the auxiliary button unit 400 is the same as mentioned above, they will be not described below.

Moreover, the sensor is disposed on the back surface of the mobile terminal case which faces the accommodation portion to detect the user's biometric information or external environmental information.

The kinds, functions, and mounted positions of the sensor are the same as those of the smart controller as mentioned above.

That is, the sensor may be a bio sensor for detecting biometric information of the user or an eco sensor for detecting external environmental information.

The processing of the detected values by the sensor, the sensing method, and the sensing button in the smart controller according to one embodiment of the present inventive concept may be applied to the mobile terminal case according to another embodiment of the present inventive concept, without any changes.

The control unit 200 functions to transmit the signal generated through the control of the input button unit and the detected value by the sensor to the mobile terminal, and the control unit 200 includes a communication unit for performing communication with the mobile terminal accommodated in the mobile terminal case 10.

Further, the signal can be transmitted and received between the mobile terminal and the mobile terminal case 10 through wireless communication such as Bluetooth, without any separate connection means.

A battery 300 functions to supply power to at least one of the input button unit 100 and the control unit 200, and in consideration of the thickness of the mobile terminal case 10, a coil type lithium or mercury battery is provided as the battery 300. Further, the battery 300 may be wirelessly charged.

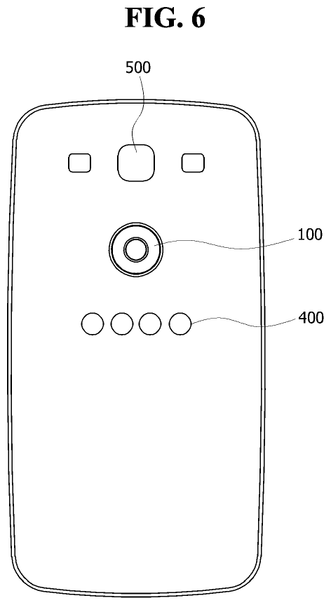

In addition to the above-mentioned parts, as shown in FIG. 6, the mobile terminal case according to another embodiment of the present inventive concept further includes a through hole 500 adapted to expose a camera lens and a light emitting diode element that are attached to the mobile terminal to the outside.

Like the smart controller according to one embodiment of the present inventive concept, the mobile terminal case according to another embodiment of the present inventive concept further includes a support unit adapted to supportingly locate the mobile terminal on a table or floor.

In specific, the support unit is rotatably coupled to the back surface of the mobile terminal case which faces the accommodation portion, and of course, it is possible that the support unit is rotatably coupled to the side surface of the mobile terminal case. An explanation of the support unit is the same as of the smart controller according to one embodiment of the present inventive concept, and accordingly, the support unit will not be described herein.

While the present inventive concept has been described with reference to the particular illustrative embodiments, it is not to be restricted by the embodiments but only by the appended claims. It is to be appreciated that those skilled in the art can change or modify the embodiments without departing from the scope and spirit of the present inventive concept.

1. A smart controller performing wireless communication with a mobile terminal and providing, to the mobile terminal, biometric information of a user of the mobile terminal or external environmental information, the smart controller comprising:
a body;
an input button unit disposed on the upper or side surface of the body to generate a control signal for the mobile terminal through the control of the user;
a sensor for detecting the biometric information of the user or the external environmental information around the user; and
a control unit disposed inside the body to receive a detection value from the sensor and to thus transmit the received detection value to the mobile terminal.

2. The smart controller according to claim 1, wherein the sensor is a bio sensor for detecting the biometric information of the user, the bio sensor being adapted to measure at least one of blood glucose, hemoglobin concentration, heart rate, body temperature, electrocardiogram, cholesterol level, and body fat rate of the user.

3. The smart controller according to claim 1, wherein the sensor is an eco sensor for detecting the environmental information around the user of the mobile terminal, the eco sensor being adapted to measure at least one of radioactive concentration, ozone concentration, carbon monoxide concentration, carbon dioxide concentration, fine dust concentration, and ultraviolet index.

4. The smart controller according to claim 1, wherein the body has an accommodation portion for accommodating the sensor therein, and the sensor is detachably attached to the accommodation portion.

5. The smart controller according to claim 1, wherein the input button unit and the sensor are formed integrally with each other.

6. The smart controller according to claim 1, wherein the mobile terminal has an application installed therein to perform at least one of functions of controlling the operation of the sensor and outputting the detection value of the sensor.

7. The smart controller according to claim 6, wherein if the operation of the sensor is requested from the user through the application, the mobile terminal transmits a sensor-operating signal to the control unit of the smart controller, and the control unit operates the sensor in response to the sensor-operating signal.

8. The smart controller according to claim 6, wherein the control unit transmits the detection value of the sensor to the mobile terminal, and based on the detection value of the sensor, the application outputs a detection result to a display of the mobile terminal.

9. The smart controller according to claim 1, wherein the input button unit comprises a sensing button for operating the sensor.

10. A mobile terminal case accommodating a mobile terminal therein and performing wireless communication with the mobile terminal, the mobile terminal case comprising:
an accommodation portion formed on the front surface thereof to accommodate the mobile terminal therein;
an input button unit disposed on the back surface of the mobile terminal case which faces the accommodation portion to generate a control signal for the mobile terminal through a user's control;
a sensor disposed on the back surface of the mobile terminal case which faces the accommodation portion to detect the user's biometric information or external environmental information; and
a control unit adapted to receive a detection value from the sensor and to thus transmit the received detection value to the mobile terminal.
